(19) United States
(12) Patent Application Publication
(10) Pub. No.: US 2015/0099988 A1
(43) Pub. Date: Apr. 9, 2015

(54) BLOOD VISCOSITY MEASURING METHOD AND SYSTEM

(71) Applicants: Andre Cernasov, Ringwood, NJ (US); Nathalie Cernasov, Ringwood, NJ (US)

(72) Inventors: Andre Cernasov, Ringwood, NJ (US); Nathalie Cernasov, Ringwood, NJ (US)

(21) Appl. No.: 14/047,792

(22) Filed: Oct. 7, 2013

(51) Int. Cl. A61B 5/02; A61B 7/04; A61B 5/00; A61B 5/0205; A61B 5/107

(52) U.S. Cl. CPC A61B 5/02035; A61B 5/0205; A61B 5/1072; A61B 5/686; A61B 7/04; A61B 5/02444
USPC 600/483; 600/500

(57) ABSTRACT

According to an example, a blood viscosity value of blood flowing through a blood vessel may be calculated by detecting a deformation of a blood vessel due to a pulsatile wave of blood flowing through the blood vessel, determining a pulsatile wave velocity of the pulsatile wave based upon the detected deformation, and calculating the blood viscosity value of the blood flowing through the blood vessel based on a predetermined relationship between blood viscosity and the determined pulsatile wave velocity.

### CLAIM FOR PRIORITY

The present application claims priority to U.S. Provisional application No. 29/910,315, filed on Oct. 5, 2012, which is incorporated by reference herein in its entirety.

### BACKGROUND

Each year cardiovascular disease claims 17 million lives worldwide, almost one million of them in the US. The main causes of death are strokes and heart attacks, both triggered by mechanical failures of the body's vascular system; more precisely by blockages in the blood flow to the brain or heart. These blockages are caused by blood clots brought about by imperfections in the blood vessels themselves, e.g., plaque deposits, stagnant blood pools, etc., by changes in blood chemistry e.g., elevated fibrinogen levels and increased platelet count, or by changes in the physical characteristics of the blood, e.g., blood viscosity and pressure. To predict the likelihood of clot formation one must monitor as many of these factors as possible.

Since the structural features of blood vessels change very slowly, tests, for example, stress tests and an echocardiogram, which may identify these structural changes, are typically performed infrequently, for example, once a year. Although blood chemistry may change faster, except in rare cases or when the blood chemistry is changed by medication, the frequency of the associated blood tests is not usually greater than a few times per year.

On the other hand, changes in the physical characteristics of blood can occur quickly. For example, even in healthy individuals, blood viscosity has been found to change throughout the day, depending on body temperature, water and food intake, and body position. Therefore, depending on a person's particular medical circumstances, meaningful blood viscosity measurements may need to be done as often as possible, ideally on a continuous basis.

At the present time, blood viscosity tests are done by off-site diagnostics labs, typically at the request of a medical doctor. The frequency of these tests ranges from once per month to once every year. Hospitalized patients may be tested at most daily. Therefore, the time between a doctor requesting the test and the lab delivering the results can vary from hours to weeks, making the timely dosing and delivery of medication difficult.

### BRIEF DESCRIPTION OF DRAWINGS

The embodiments are described in detail in the following description with reference to the following figures. The figures illustrate examples of the embodiments.

FIG. 1 illustrates a block diagram of a blood viscosity measuring system according to an example of the present disclosure.

FIG. 2 illustrates an implantable dual-sensor sensing module according to the system depicted in FIG. 1 according to an example of the present disclosure.

FIGS. 3a-3d, collectively, illustrate an operation of the dual-sensor sensing module depicted FIG. 1 according to an example of the present disclosure.

FIG. 4 illustrates an implantable single sensor sensing module according to the system of FIG. 4 according to an another example of the present disclosure.

FIGS. 5a-5c, collectively, illustrate the operation of a single sensor sensing module according to the system of FIG. 4 according to an example of the present disclosure.

FIG. 6 illustrates an implantable sensing module according to either of the systems depicted in FIGS. 1 and 4 according to an example of the present disclosure.

FIG. 7 illustrates a flowchart depicting operation of the dual-sensor implantable sensing module depicted in FIG. 1, according to an example of the present disclosure.

FIG. 8 illustrates a flowchart depicting operation of the single-sensor implantable sensing module depicted in FIG. 4, according to an example of the present disclosure.

FIG. 9 illustrates a flowchart depicting an operation of a non-implantable blood viscosity measuring system, according to an example of the present disclosure.

FIG. 10 is an environmental drawing of the non-implantable dual-sensor blood viscosity measuring system depicted in FIG. 9 according to an example of the present disclosure.

FIG. 11 illustrates a flowchart depicting an operation of the non-implantable blood viscosity measuring system depicted in FIG. 9 according to an example of the present disclosure.

FIG. 12 illustrates a block diagram of the non-implantable dual-sensor blood viscosity measuring system depicted in FIG. 9 according to an example of the present disclosure.

FIG. 13 is an environmental drawing of another non-implantable dual-sensor blood viscosity measuring system, according to another example of the present disclosure.

FIG. 14 illustrates a block diagram of the non-implantable dual-sensor blood viscosity measuring system depicted in FIG. 12, according to an example of the present disclosure.

FIGS. 15a-5c, collectively, illustrate a wrist-worn dual-sensor blood viscosity measuring system according to an example of the present disclosure.

### DETAILED DESCRIPTION

For simplicity and illustrative purposes, the principles of the embodiments are described by referring mainly to examples thereof. In the following description, numerous specific details are set forth in order to provide a thorough understanding of the embodiments. It is apparent that the embodiments may be practiced without limitation to all the specific details. Furthermore, the embodiments may be used together in various combinations.

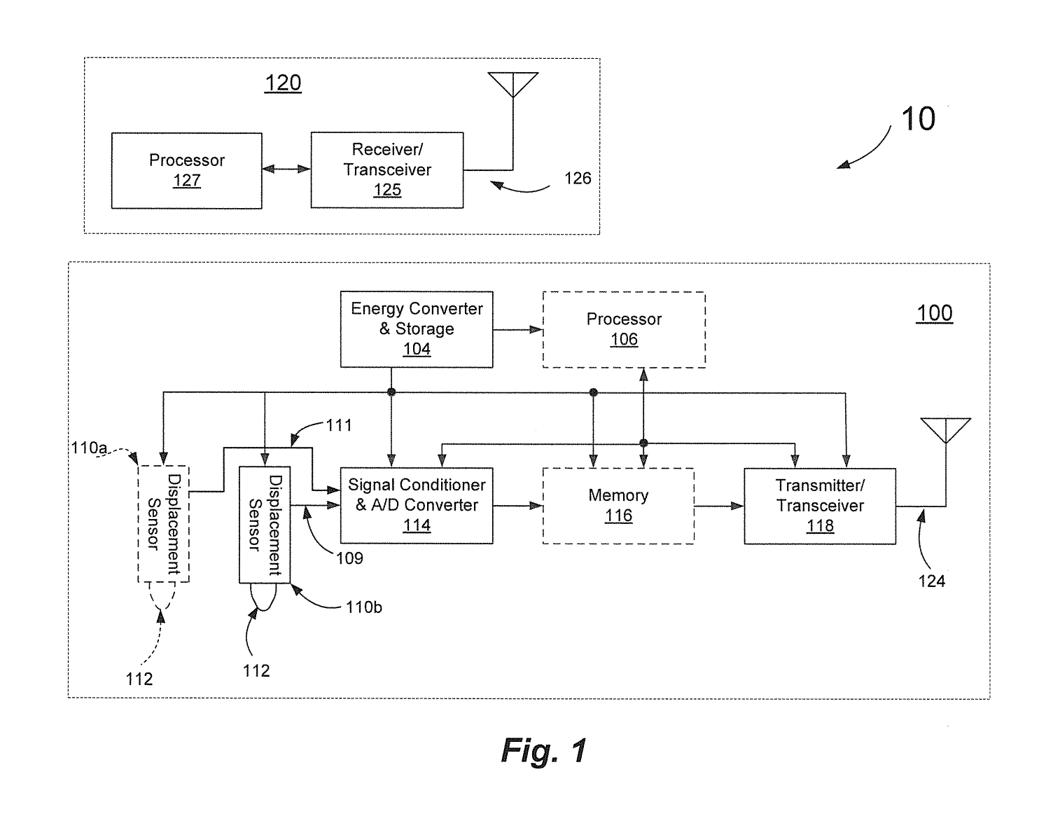

FIG. 1 depicts one embodiment of a system 10 to continuously monitor blood viscosity, and includes a sensing module 100 and a processing unit 120.

The viscosity of human blood at 37° Celsius is normally in the range of 3 to 4 centipoise. To monitor blood viscosity, system 10 measures the speed of travel (velocity) of a pulsatile wave that propagates in most blood vessels with each contraction of the heart.

### Principles of Operation

Speed of propagation of blood through a blood vessel may be modeled using the following equations that are used to calculate the speed of a pulsatile wave through a pipe. (See “Pulsatile blood flow in the entire coronary arterial tree: theory and experiment”, Yunlong Huo and Ghassan S. Kassab, Am J Physiol Heart Circ Physiol 291:H1074-H1087, 2006, the disclosure of which is incorporated by reference in its entirety).

$$\begin{matrix}V ( ω ) = {V}_{0} \sqrt{1 - F ( α )} & ( 1 ) \\ F ( α ) = [ 2 {J}_{1} ( {i}^{3 / 2} α ) ] / [ {i}^{3 / 2} {αJ}_{0} ( {i}^{3 / 2} α ) ] & ( 2 ) \\ α = R \sqrt{ωρ / η} & ( 3 ) \\ {V}_{0} = \sqrt{\frac{Eh}{2 ρ R}} & ( 4 )\end{matrix}$$

In regard to a blood vessel, V(ω) is the frequency component of the longitudinal pulsatile wave velocity; V₀ is the longitudinal velocity of the pulsatile wave in an inviscid fluid of the same density travelling through the blood vessel; R the inner radius of the relaxed blood vessel, i.e., at the low point of blood pressure; ρ is the density of the fluid; ω is the frequency component of the velocity being observed; η is the viscosity of the blood travelling through blood vessel; α is the Womersley number which is a dimensionless expression of the pulsatile flow frequency in relation to viscous effects. E is the Young's modulus of the vessel wall, h is the wall thickness, and J₀ and J₁ are Bessel function of the first kind, and order zero and one, respectively, and i is the unit imaginary number (\sqrt{−1}).

Although several of the above parameters may vary significantly from individual to individual, typical numbers for a medium size artery may be as follows: h=0.1 cm; R=0.2 cm; ρ=1 g/cm³; and E=10⁷ dynes/cm². Based upon the above-parameters, the inviscid velocity for a medium size artery is approximately V₀=1600 cm/sec=16 m/sec.

For a first order analysis, ω may be assumed to be evaluated as the dominant frequency, i.e., the heart rate at frequency f=HR and thus, ω=2πHR.

At a heart rate (HR) of 60 beats per minute (1 Hz) α may be approximated as:

$$α = \frac{1}{2 \sqrt{η}} = 0.5 {η}^{- 1 / 2} \qquad ( 5 )$$

In addition, the velocity of the pulsatile wave at the heart rate may be calculated through,

V(HR)=1600\sqrt{1−[4J₁(0.5i^{3/2}η^{-1/2})]/[i^{3/2}η^{-1/2}J₀(0.5i^{3/2}η^{-1/2})]} (6)

Thus, based upon a measured value of the longitudinal pulsatile wave velocity at a subject's heart rate, V(HR), the blood viscosity η may be determined through implementation of a known numerical method, which may be stored in memory 116 as machine readable instructions.

Equations (1)-(6) clearly demonstrate the dependency of the longitudinal pulsatile wave velocity on blood viscosity. Therefore, by measuring a subject's longitudinal pulsatile wave velocity V(HR), system 10 may calculate the subject's blood viscosity.

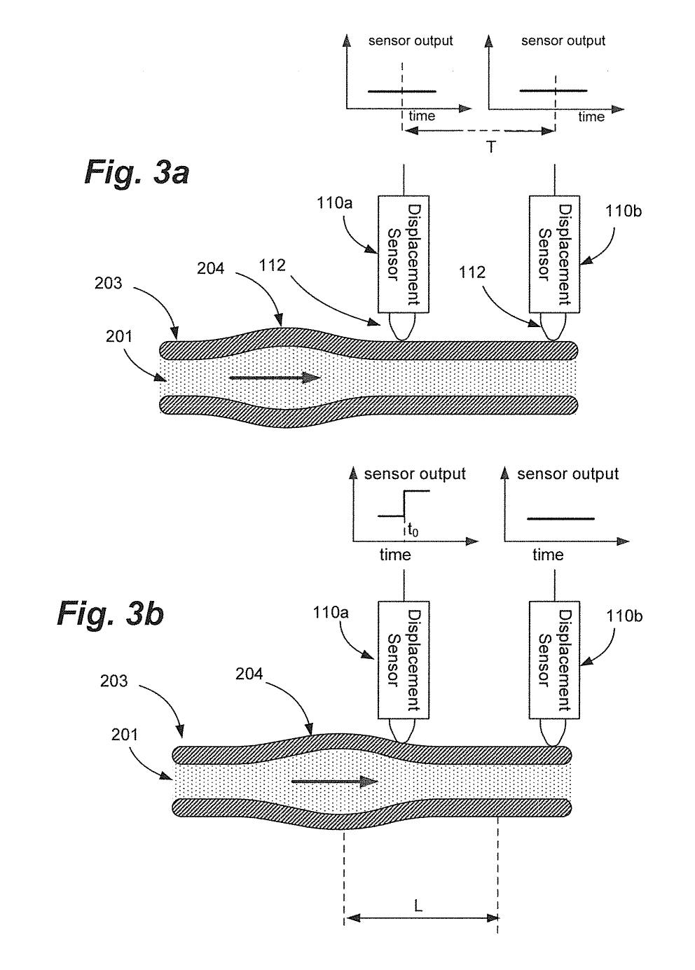

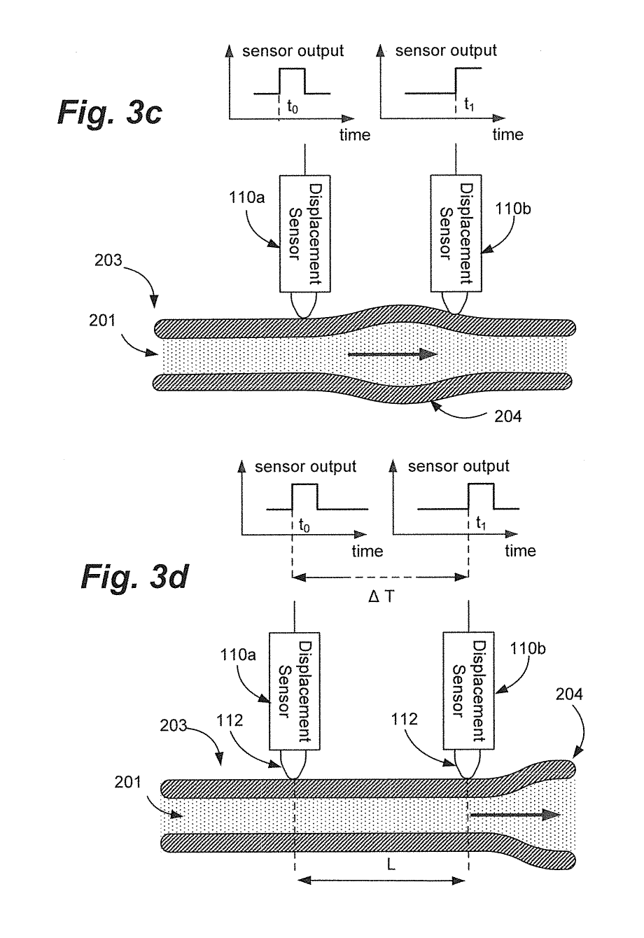

The longitudinal pulsatile wave velocity may be derived by measuring the time of arrival of a pulsatile wave front, i.e., bulge, at two wave front sensor locations separated by a known distance L, as shown in FIGS. 3a-3d. If the arrival time at sensor 110a is t0 and the arrival time at sensor 110b is t1, the longitudinal pulsatile wave velocity may be calculated as follows:

$$V ( HR ) = \frac{L}{t 1 - t 0} \qquad ( 7 )$$

By replacing V(HR) in equation (6) with the expression in equation (7) and numerically inverting equation (6), various disclosed embodiments of a disclosed blood viscosity measuring system may obtain the blood viscosity value q.

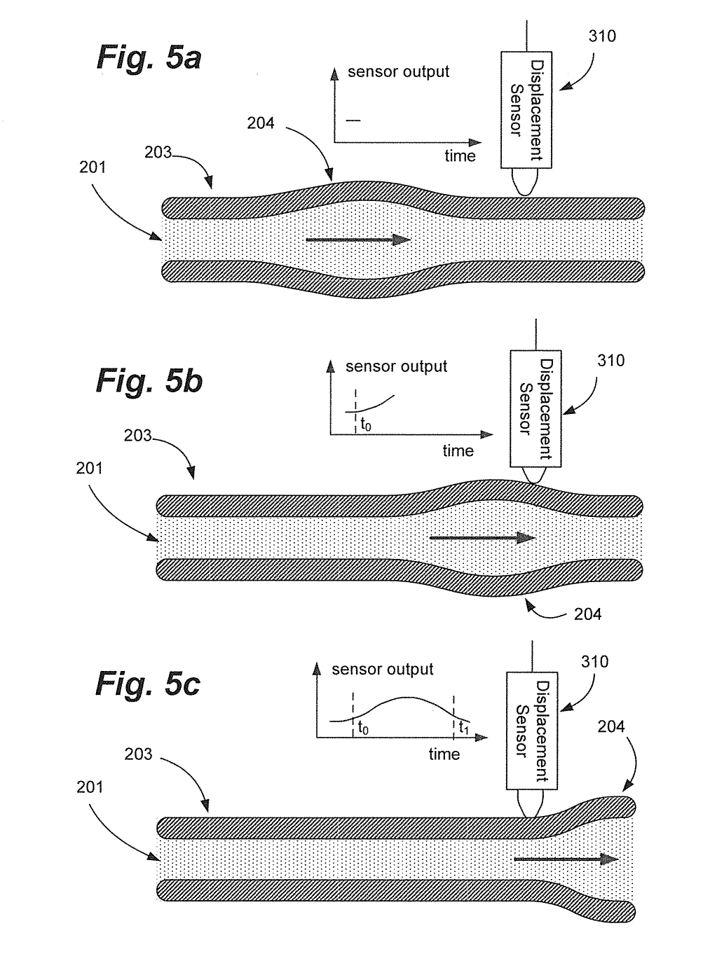

FIGS. 5a-5c illustrate an alternative method and system for determining the longitudinal wave velocity using a single high resolution displacement sensor to map the profile of the bulge as it passes by a displacement sensor. According to an example, an analysis of the blood viscosity level may be restricted to when the body is at steady state rest and the heart stroke volume is constant, and taking the assumption that the share of the stroke volume allocated to the bulge within the vessel of interest is also constant, to thus enable an approximation of the total volume of the bulge to be a constant, V_{B0}.

Thus, by detecting the bulge at t0 and detecting the bulge at t1, and by determining a profile function y(t) of the bulge as a function of time, the velocity of the pulsatile wave at frequency of the heartbeat, V(HR), may be calculated to be the inverse of:

$${V}_{B 0} = π V ( HR ) {\int }_{{t}_{0}}^{{t}_{1}} {y}^{2} [ t , V ( HR ) ] d t \qquad ( 8 )$$

For an approximation where, between t0 and t1, the wave front, or bulge, is a cylinder of radius RB V(HR) may be defined using equation (9), as follows:

$$V ( HR ) = \frac{{V}_{B 0}}{π {R}_{B}^{2} ( {t}_{1} - {t}_{0} )} \qquad ( 9 )$$

Different embodiments may use more complex models for the pulsatile bulge such as polynomial approximations.

Once V(HR) is determined, the numerical inverse of equation (6) provides the value of the blood viscosity q.

The determined blood viscosity level based upon the preceding equations is an approximate level and is based upon a number of limiting assumptions. Other embodiments of the blood viscosity measuring system using the sensors and other components described herein may be based upon more detailed relationships between blood viscosity, blood vessel parameters and the radial expansion of blood vessel. Such detailed relationships may be found in “The Physics of Pulsatile Flow” by M. Zamir, Springer, ISBN: 0-387-98925-0, which is incorporated by reference herein in its entirety.

The disclosed blood viscosity measuring devices provide continuous or on-demand measurements that can be stored and processed in real time or off-line.

Sensing module 100 and processing unit 120 may be incorporated in a single unit or may be included in separate modules in wireless communication with each other. In at least one embodiment, sensing module 100 is implantable in a user's body and is attached so as to surround an arterial vessel exhibiting pulsatile wave motion. When the sensing module 100 is implanted in a user's body, processing unit 120 may be external to the user's body.

In FIG. 1 sensing module 100 includes at least one displacement sensor 110 to detect displacement, i.e., a bulge, in the wall of a blood vessel due to a pulsatile wave of blood passing through the blood vessel. Although embodiments described herein depict one or two sensors 110a and 110b, additional sensors may be incorporated. Sensors 110a and 110b transmit displacement signals 109 and 111, respectively, to a Signal Conditioner and Analog to Digital (A/D) converter 114. Displacement sensors 110a and 110b may detect inductive, capacitive, resistive, piezoelectric, photoelectric or other known variation based upon a displacement of a wall of a blood vessel.

Under control of a processor 106, Signal Conditioner & A/D Converter 114 converts the output signals 109, 111 of the displacement sensors 110a and 110b into digital format and in at least one embodiment, calculates a blood viscosity value. The calculated blood viscosity value and/or the digitized displacement measurements may be forwarded to a wireless transmitter/transceiver 118, which may transmit the calculated blood viscosity value and/or the digitized displacement measurements to a receiver/transceiver 125 via antennas 124 and 126. The receiver/transceiver 125 may also forward the calculated blood viscosity value and/or the digitized displacement measurements to a processor 127 of processing unit 120.

In at least one embodiment, processor 106 is an ultra-low power processor of the type used in implanted pacemakers and defibrillators. The wireless transmitter/transceiver 118 is an ultra-low power on demand RF device that communicates with processing unit 120 via a wireless link which may be RF, IR acoustic or other wireless communication technology including BLUETOOTH™. The data transmission may be periodic, continuous, pushed to or pulled from processing unit 120.

Processing unit 120 may, in some embodiments, communicate with processor 106 of module 100 to provide software updates and other information.

Memory device 116 is a non-volatile local memory to store program code executed by the processor 106 as well as to store the values of the displacement signals 109, 111 prior to being communicated to the processing unit 120.

Furthermore, as presented above, the calculated blood viscosity for a specific blood vessel at a specific location in the body is based upon structural and dimensional parameters of the particular blood vessel being sensed. However, once these parameters are determined, they are relatively stable. For implantable devices these parameters may be measured directly at the time of surgical implantation using conventional tools and techniques.

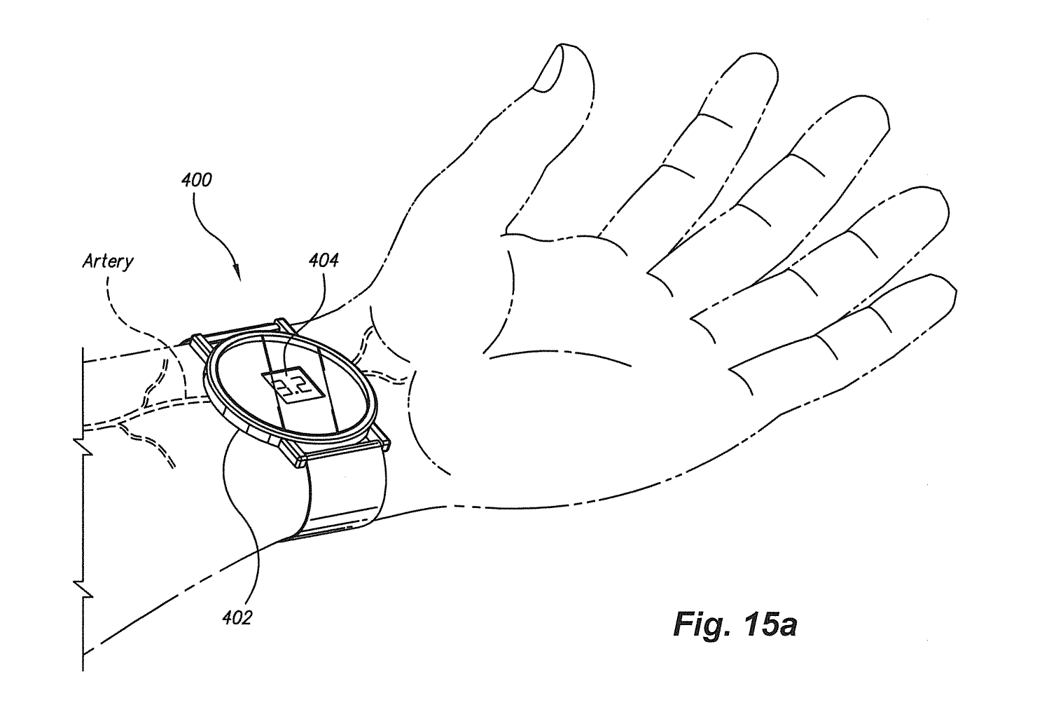

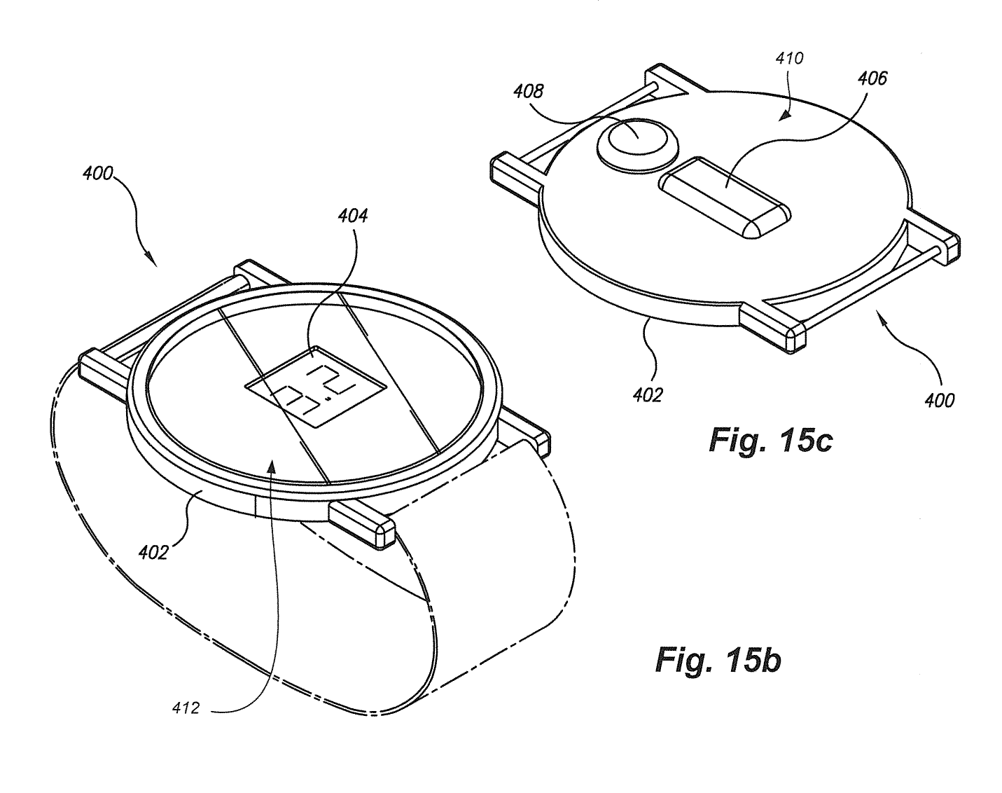

For a wearable device, such as depicted in FIGS. 15a-c, these parameters may be calculated from data obtained from an initial blood viscosity measurement using blood drawn from the same blood vessel to be sensed by device 400. Periodic reference measurements, made using standard laboratory devices such as “falling body”, “cone and plate” or Ostwald U-tube viscometers, may be implemented to recalibrate both the implanted and wearable devices disclosed herein as the condition of the body may change with age, health, diet, etc. In order to provide an implantable device or wearable device with the calibration data necessary to compute blood viscosity, processing unit 120 may be used to transmit this data to processor 106 of these devices.

According to an example, electrical energy to power the components of the sensing module 100 may be provided by an energy converter and storage device 104, which may include a mechanical-electrical device that converts some of the energy contained in the pulsatile motion of a vascular vessel into electrical energy, and a capacitor or other energy storage device to store the electrical energy to power sensing module 100. In another example, the energy converter and storage 104 may instead be a battery.

The complexity of processor 106 and the size of memory 116 depend on the amount of energy available. When sensing module 100 is placed around a larger vessel more energy may be available to sensing module 100. When sufficient power is available, processor 106 may calculate the blood viscosity value and communicate the calculated value to the processing unit 120 on a non-continuous basis at a predetermined time or in case of an emergency.

FIG. 1 additionally illustrates an optional low-power embodiment. As indicated by dashed lines around processor 106 and memory 116, outputs 109 and 111 of the displacement sensors 110a and 110b may be transmitted directly via transmitter/transceiver 118 to the processing unit 120 with minimal conditioning. In this low-power embodiment, processor 106 and memory 116 are omitted to reduce power requirements and the calculation of blood viscosity is performed in processing unit 102.

As presented above, processing unit 120 and sensing module 100 include processor 106 and 127, respectively, which implement or execute machine readable instructions performing some or all of the methods, functions and other processes described herein.

Processor 127 of processing unit 120 may range in sophistication from a dedicated medical workstation that includes multiple components, e.g., a display device and interfaces to external systems, to a smart phone or tablet computing device. In some embodiments, processing unit 120 receives and processes the information provided by sensing module 100. In embodiments in which sensor module 100 directly calculates blood viscosity values, processing unit 120 may provide storage, reporting, and display functions allowing the calculated blood viscosity value and other data to be presented to, for example, the user.

The methods, functions and other processes described herein may be embodied as machine readable instructions stored on a computer readable medium, which may be non-transitory, such as hardware storage devices (e.g., RAM (random access memory), ROM (read only memory), EPROM (erasable, programmable ROM), EEPROM (electrically erasable, programmable ROM), hard drives, and flash memory).

Embodiments of the disclosed blood viscosity measuring system may be either implantable in a user's body, or may be worn by the user with at least one sensor pressed against the skin of the user. Regardless of whether the blood viscosity measuring device is implanted or is externally worn, blood viscosity values may be calculated in real-time.

### Implantable Embodiments

Implantable embodiments of a blood viscosity measuring system include one or more subcutaneous sensors, and may apply different sensor types and computational methods to measure pulsatile wave velocity and blood viscosity based upon the number and type of sensors.

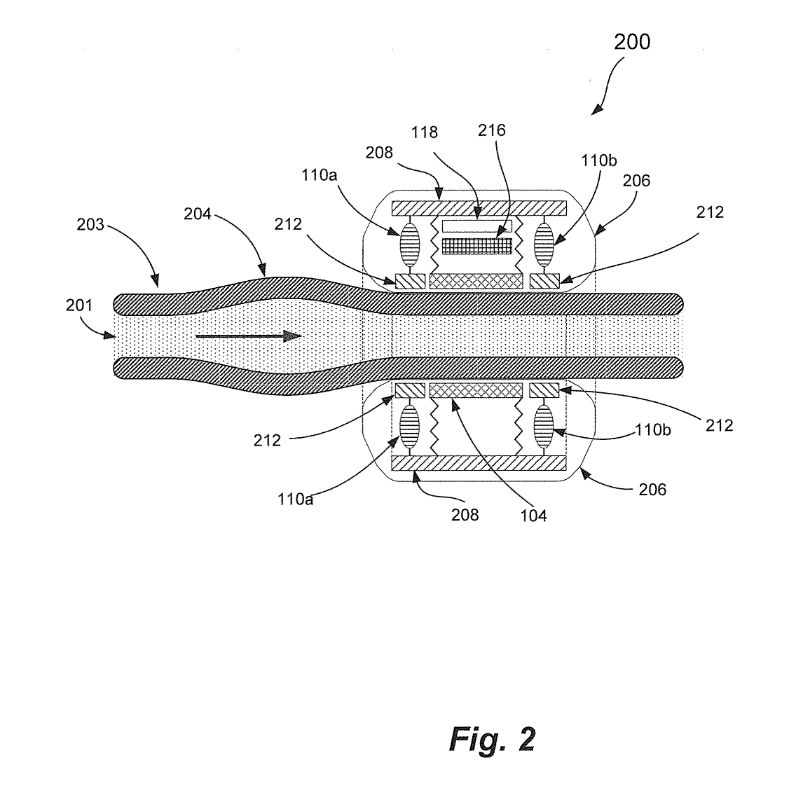

FIGS. 2 and 3 depict an implantable embodiment 200 of sensing module 100 that includes displacement sensors 110a and 110b placed a known distance apart from each other. In one embodiment, sensors 110a and 110b are placed approximately ¼ inch apart. In addition, in this embodiment, processor 106, memory 116, and Signal Conditioner & A/D Converter 114 are integrated into a single component 216.

Sensing module 200 further includes a flexible ring 206 to surround a blood vessel 203 that experiences periodic propagating pulsatile movements caused by a flow of vascular fluid (i.e., blood) 201. Blood vessel 203 may be one of the many large or medium size blood vessels making up a person's arterial vascular system and exhibits a pulsatile motion of measurable amplitude. For reference purposes, the flow of blood 201, and therefore the pulsatile motion, is depicted as propagating from left to right.

Ring 206 provides structural support for imbedded components of sensing device 200 and presents a barrier protecting blood vessel 203 from direct contact with the imbedded components and further prevents the components from coming in direct contact with interstitial fluids. Ring 206 may be made of a soft, flexible, elastic, compressible, biocompatible, long-life material, which may easily be compressed under the pressure of a wave front, or bulge 204, formed by the pulsatile wave. All the components of implantable sensing device 200 may be embedded within ring 206. Ring 206 may incorporate a rigid outer portion 208 against which the elastic body of ring 206 compresses. Outer ring portion 208 may form a reference structure forming a basis for signals detected or generated by sensors 110 and energy converter 104.

In the implantable dual-sensor sensing module 200, coupling shoes 212 and energy converter 104 may be spaced apart at a known distance L and may be mounted proximate to a wall of vessel 203 through ring 206. The greater the distance L, the more accurate will be the calculated viscosity value.

Coupling shoes 212 may each transmit a measure of displacement of the wall of vessel 203 caused by bulge 204 to displacement sensor 110a and 110b. Reference sides of sensors 110a and 110b may be fixed to the rigid outer ring 208.

Furthermore, in the implantable version of sensing module 200 the energy converter and storage device 104 may be of the type described in U.S. Pat. No. 7,813,810, the disclosure of which is incorporated by reference in its entirety. Because power may only be generated when the wave propagates through the implanted sensing device 200, a local energy storage device, such as a capacitor, may be used to store the energy. For a wearable version of the blood viscosity measuring device, other power systems, such as batteries and inertial energy collecting devices, may also be used.

As shown in FIGS. 2 and 3a-3d, sensors 110 may be positioned to measure a displacement of the wall of blood vessel 203 caused by bulge 204. Under control of processor 106, sensing module 200 may directly calculate a blood viscosity value and may output the calculated blood viscosity value to a user via processing unit 120. As previously mentioned, in low-power embodiments, only the displacement measurements 109, 111, or conditioned versions of the displacement measurements, may be transmitted to the processing unit 120, which may then calculate the blood viscosity value. Because sensing module 200 is placed against or around a blood vessel, sensing device 200 does not come in contact with the blood in the blood vessel and therefore does not produce secondary effects such as obstruction induced clotting.

As shown in FIGS. 3a-3d, a wave of fluid 201 propagates through blood vessel 203 causing a bulge 204 in the wall of vessel 203. The bulge 204 first reaches displacement sensor shoe 112 that triggers sensor 110a, which transmits a first signal 111 to processor 106. The wave then reaches shoe 112 of sensor 110b, which generates and transmits a second input signal 109, to processor 106.

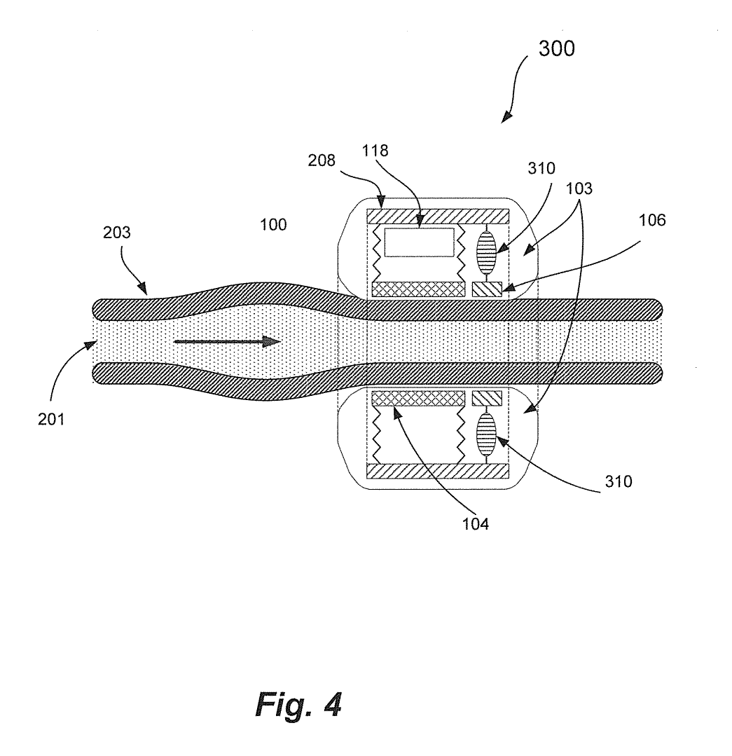

FIGS. 4, 5a-5c, and 6 depict an implantable sensing module 300 having a single more complex displacement sensor 310 to take a burst of measurements of the shape of the pulsatile wave as the pulsatile wave propagates through sensing module 300. A method of calculating blood viscosity according to the single sensor of sensing module 300 may include determining a profile function y(t) of the bulge 204 as a function of time, and finding the numerical inverse solution to the system of equations (6) and (8).

In the “sensor only” embodiment, that is, in a sensing module 100 that includes only at least one sensor and does not include processor 106 or memory 116, or in a sensing module 100 in which processor 106 provides minimal functionality, the blood viscosity value may be separately calculated by processing unit 120.

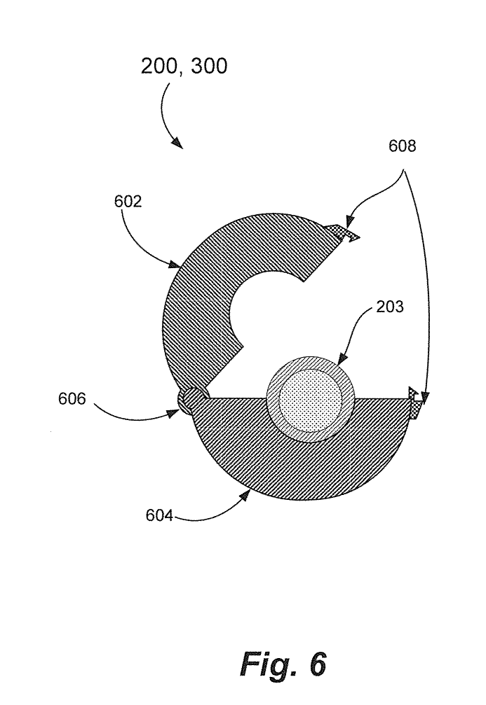

FIG. 6 illustrates an implantable sensing module 200, 300 placed around arterial blood vessel 203, according to an example. For proper operation, the implantable version of sensing module 100 may need to substantially surround vascular vessel 203. In order not to require severing blood vessel 203, FIG. 6 illustrates a sensing module 200, 300 that may be split into two half rings 602 and 604 connected on one side by a hinge 606 and on the other side by a latch 608. Sensing module 200, 300 may be placed around blood vessel 203 by opening the latch 608, rotating the half rings around the hinge 606, placing half rings 602 and 604 around the vessel 203, and closing the latch 608. In an alternative design, half rings 602 and 604 may be completely, detachable and may be snapped together using two latches 608.

### Implantable Dual-Sensor Operation

In order to measure blood viscosity using equations (1) through (9) or other valid solutions, the velocity of the pulsatile wave that propagates through the blood vessel with each heartbeat must be calculated. In the multiple sensor configurations disclosed herein, the velocity may be obtained by measuring the time interval (ΔT=t1−t0) required for a wave front to traverse a known distance L, or an equivalent distance Leq.

As depicted in at least FIG. 3d, the velocity of the blood 201 flow may be calculated by dividing the space separation L between the shoes 112 of the two sensors 110a and 110b by the time difference ΔT between the signals 111, 109 generated by sensors 110a and 110b as the pulsatile wave propagates starting from the heart and eventually arrives at sensors 110a and 110b.

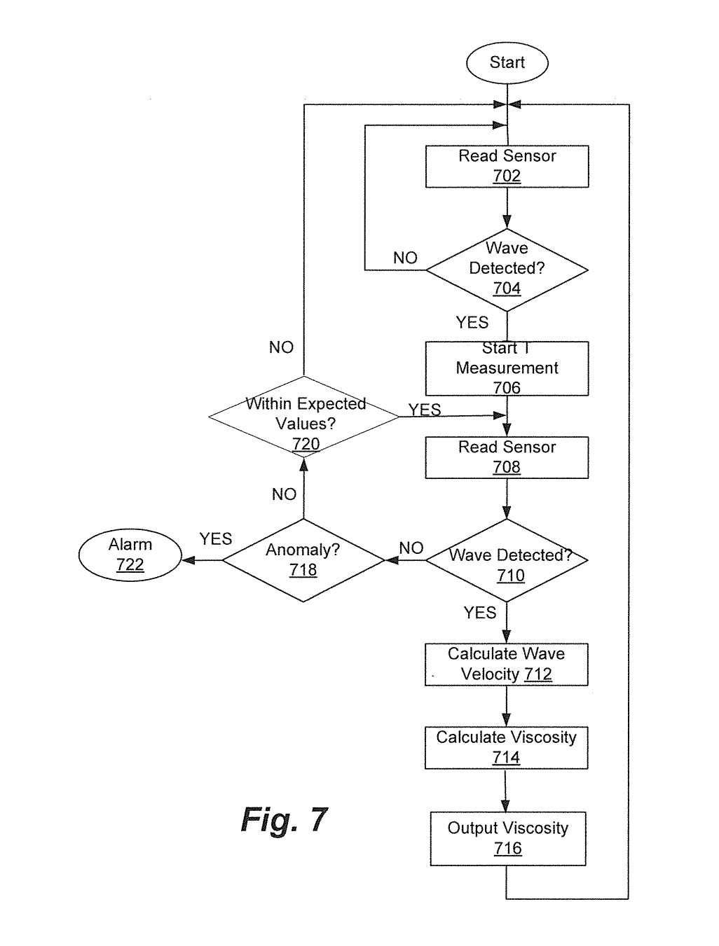

FIG. 7 depicts a flowchart illustrating a representative process for calculating blood viscosity in a dual-sensor sensing module depicted in FIG. 1, according to an example.

In an idle mode, at blocks 702 and 704, the sensing device may continuously wait for the leading edge of a pulsatile wave to reach a first displacement sensor 110a. Upon detection of the leading edge of the wave, sensing module 100 may initiate a timer, implemented in hardware or machine-readable instructions, at block 706. At blocks 708 and 710, the leading edge may be detected at sensor 110b and the time interval (ΔT) required for the wave to travel from sensor 110a to sensor 110b may be determined.

If a pulse is registered by first sensor 110a but not by second sensor 110b within a predetermined amount of time, the system enters an analysis mode at block 718, to determine whether the problem is due to an anomaly (malfunction, unusual vascular events, etc.) or is due to noise (within expected values). In response to a determination that the problem is due to an anomaly sensing module 100 may transmit or register an alarm condition at block 722.

In response to a determination at block 720 that the anomaly is due to noise, the sensing module 100 may be reset and may return to the idle mode. Otherwise, control may be transferred to block 708 to wait the detection of the wave by second sensor 110b.

At block 712, in response to second sensor 110b detecting the bulge within an expected time period, the velocity of the pulsatile wave is calculated by dividing the distance L between sensors 110 by time T.

At block 714, machine-readable instructions stored in either memory 116 or in processing unit 120 may be executed by at least one of processor 106 and 127 to calculate the blood viscosity value based as discussed above.

At block 716, system 10 may output the calculated blood viscosity value to a user via a display device or other reporting device.

### Implantable Single Sensor Operation

As shown in FIGS. 5a to 5c, the speed of propagation of the pulsatile wave may also be inferred from the shape of the deformation bulge 204 of blood vessel 203 at the front of the wave. The faster the wave travels the faster the blood vessel 203 will swell and recover. In the single sensor configuration shown in FIGS. 4 and 5a-5c, sensing module 300 may take multiple measurements of each bulge 204 using a single sensor 310 to profile the shape of the wave. From these multiple measurements, processor 106 or processor 127 may calculate the speed of propagation of the pulsatile wave (FIG. 1). Viscosity of the blood may then be calculated as discussed above using software instructions stored in memory and executed by a processor.

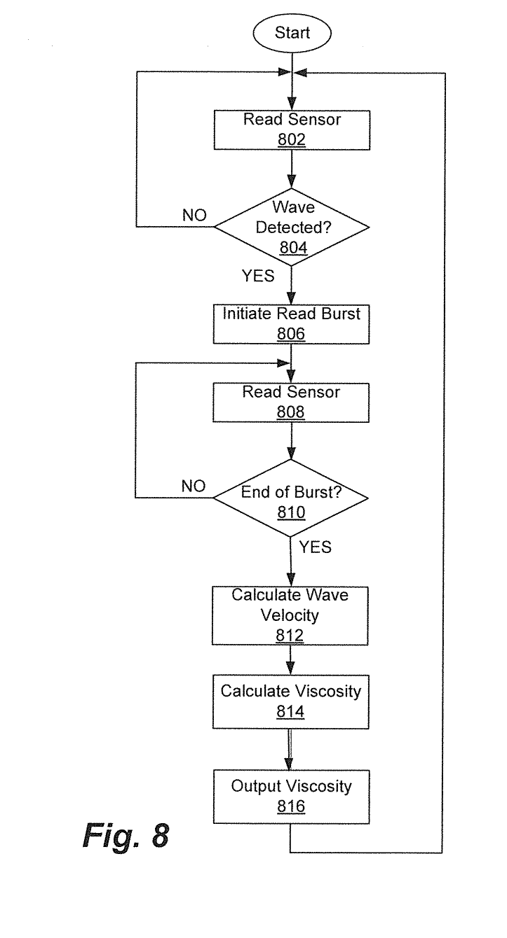

FIG. 8 depicts a flowchart executed by sensing module 300 to calculate blood viscosity in a single sensor configuration according to an example.

Blocks 802 and 804 may be an idle state in which processor 106 waits for a leading edge of the pulsatile wave to reach, and be detected, by sensor 310.

At block 806, and upon detection of the leading edge of the pulsatile wave, machine instructions cause processor 106 or processor 127, to initiate capturing by sensor 310 of a burst of measurements to allow profiling of the bulge 204 in blood vessel 203. At block 808, the sensor 310 may capture the burst of measurements. At block 810, sensing module 300 waits for the diastolic pause before calculating, at block 812, the pulsatile wave velocity.

At block 814, the blood viscosity value may be calculated based upon equations (1) through (9) or other valid solutions, by at least one of processors 106 and 127.

At block 816, the value of the blood viscosity may be outputted to a suitable output device, for example, a LED or LCD display.

### Non-Implanted Embodiments

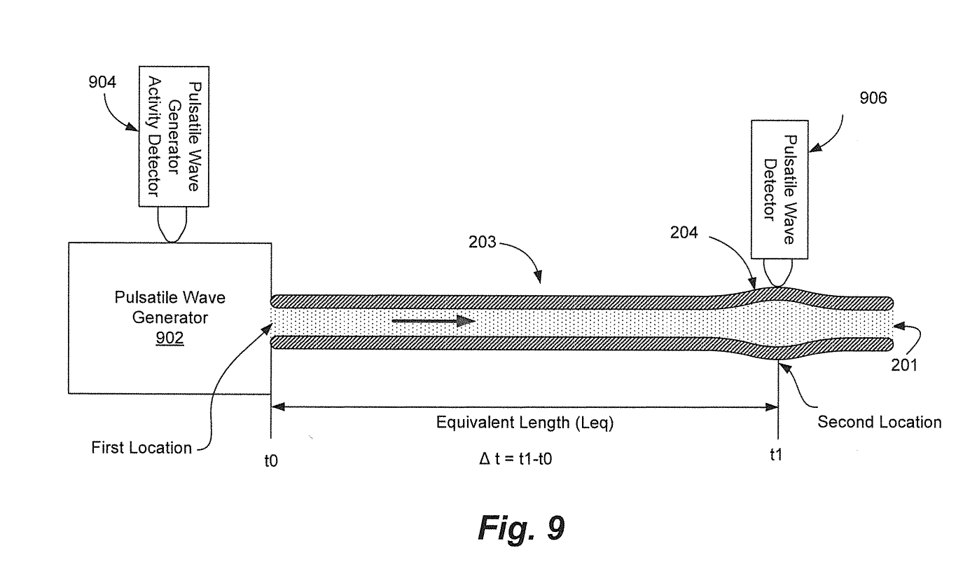

FIG. 9 illustrates a non-implanted embodiment of the blood viscosity measuring system that includes a first sensor 904 positioned in the proximity of a pulsatile wave generator 902, for example a person's heart, and a second sensor 906 positioned at a location having a known equivalent length (Leq) away from the heart.

In the process of generating a pulsatile wave, the heart may produce a number of signals that are non-displacement type signals. For example, the heart may generate an audible sound, i.e., a heartbeat, or a characteristic electrical signature which accompanies the generation of a pulsatile wave by the heart.

First sensor 904 may be placed in the proximity of the heart, on a chest strap, for example, to detect time t0. First sensor 904 may measure the sound of the heartbeat or the characteristic electrical signature that accompanies the generation of a pulsatile wave. First sensor 904 may be a microphone, an electrometer, a piezoelectric sensor, a photoelectric sensor or other sensor specific to the characteristics of the signal to be detected. In any event, first sensor 904 may detect the signal generated by the heart and may identify this signal as being generated at time t0.

Second sensor 906 is a displacement sensor to detect the bulge of the pulsatile wave as the pulsatile wave is propagated from the heart to the wrist and may operate to detect a pulsatile displacement at time t1. Second sensor 906 may be held against the skin of the user's wrist by a wristband. The second sensor 906 may detect the bulge through implementation of either or both of acoustic and photoelectric technologies.

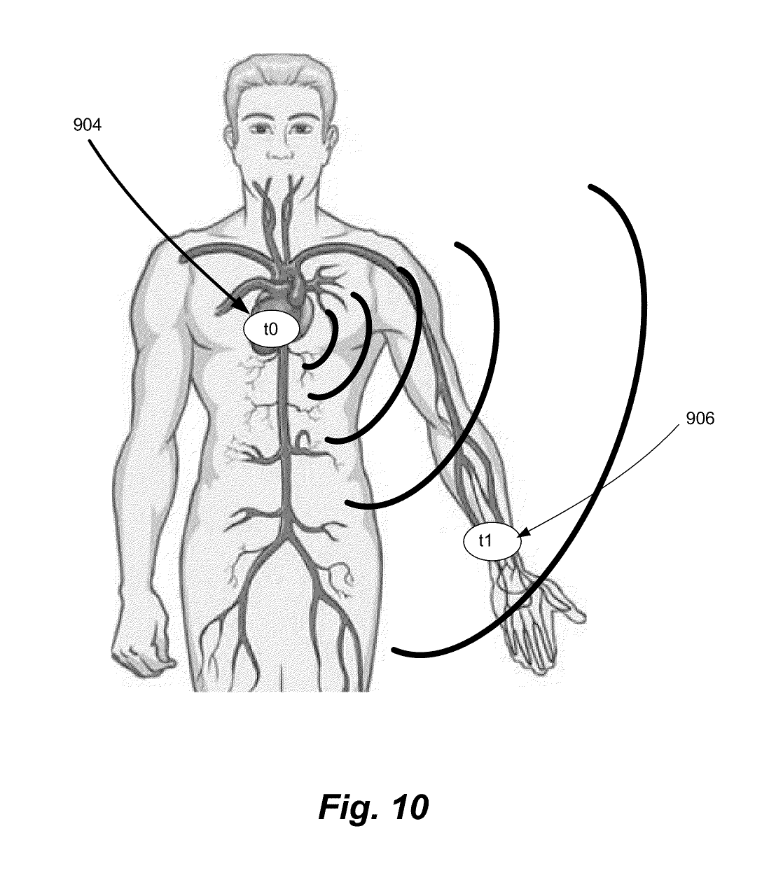

When calculating the velocity of propagation of a pulsatile wave between the heart and a point of measurement downstream of a blood vessel, the blood viscosity measuring system may account for the fact that the two locations are not connected by a continuous vessel but rather by the arterial tree illustrated in FIG. 10. Thus, the non-implantable embodiments described herein may utilize an artificial construct of an equivalent vessel having a length Leq between the heart and the point of measurement and exhibits the same velocity-viscosity characteristic as the arterial tree.

The length Leq of the equivalent vessel, may be determined when initially calibrating the system 10. Using, for example, a laboratory measurement of the viscosity of the blood at the point of measurement, and a determined t1−t0, V(HR) may be calculated using equation (6). Solving equation (7) for Leq results in equation (10) as follows.

Leq=(t1−t0)V(HR) (10)

Thus, once Leq is known, the blood viscosity measuring device may then use Leq in subsequent real-time calculations of V(HR). The blood viscosity measuring device may then apply the inverse of equation (6) to determine a real-time blood viscosity value.

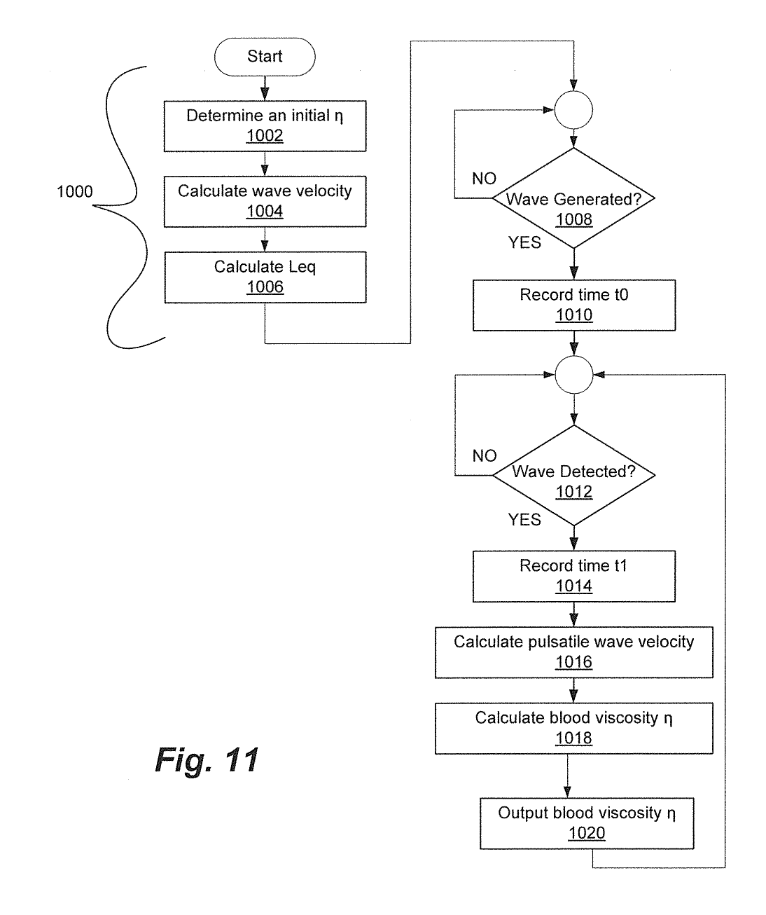

FIG. 11 depicts a flowchart to calculate blood viscosity in a non-implantable blood viscosity measuring device, according to an example.

At an initial calibration stage 1000, the process may include the following functional blocks.

Measuring, at block 1002, an initial blood viscosity at a location of the sensor based upon an analysis of blood taken at the location of the sensor. The initial blood viscosity may be measured by obtaining a blood sample and measuring the blood viscosity of the blood sample.

Calculating, at block 1004, a velocity of the pulsatile wave based upon the measured blood viscosity, using for example, equation (6) that relates velocity of the blood to the viscosity of the blood.

Calculating at block 1006, an equivalent arterial distance, Leq, based upon the calculated velocity of the pulsatile wave and a determined time difference between the detected deformation of the blood vessel and a detected signal relating to the generation of the pulsatile wave.

Once the equivalent arterial distance, Leq, is determined and stored in memory, the device may subsequently determine real-time values of blood viscosity by: detecting, at block 1008, a signal related to the generation of a pulsatile wave by the heart, recording, at block 1010, of the time t0 when the pulsatile wave was generated, detecting, at block 1012, a deformation of the arterial vessel related to the pulsatile wave generated at time to, recording, at block 1014, the time t1 when the arterial vessel deformation was detected, calculating, at block 1016, the pulsatile wave velocity based upon Leq, t1 and t0, and calculating blood viscosity q, at block 1018.

At block 1020, the calculated blood viscosity q may be outputted to a user. The process may then continue to automatically monitor and re-calculate blood viscosity q or wait for initiation by the user.

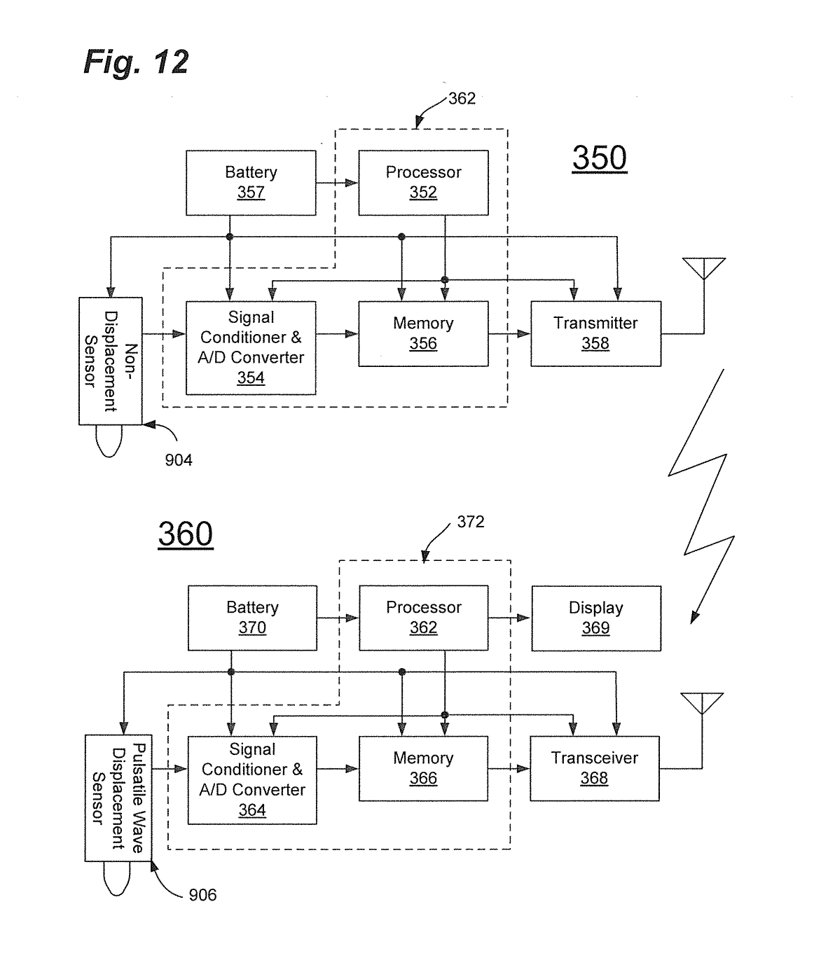

FIG. 12 depicts a block diagram of a first sensor module 350 and includes a non-displacement sensor 904 that detects heart activity associated with the generation of a new pulse. For the human heart, such activity may be, for example, a characteristic sound or an electrical signal. Module 350 further includes a controller module 362 that further includes a processor 352, a signal conditioner and A/D converter 354 to make the sensor measurement readable by processor 352, and a memory 356 to store at a minimum, instructions readable by processor 352. Module 350 further includes a transmitter 358 which may transmit the time t0 of when each pulsatile wave is generated to a second sensor module 360.

Second sensor module 360 may include one or more displacement sensors 906 to directly detect displacement of the blood vessel at, for example, a user's wrist, at time t1 due to the pulsatile wave. Module 360 is further depicted as including a controller module 372, a transceiver 368 and a display device 369. Transceiver 368 may receive signal t0 from transmitter 358. The time delay between the transmission and receiving of time signal t0 may be assumed to be negligible, or may be taken into account by the program executed by processor module 372.

Similar to controller module 362, controller module 372 may include a signal conditioner and A/D converter 364, a processor 362, and a memory 366. Signals detected by displacement sensor 906 and the signals received by transceiver 368 are processed by processor 372 to calculate the blood viscosity level. The calculated blood viscosity level may be outputted, for example, on display device 369.

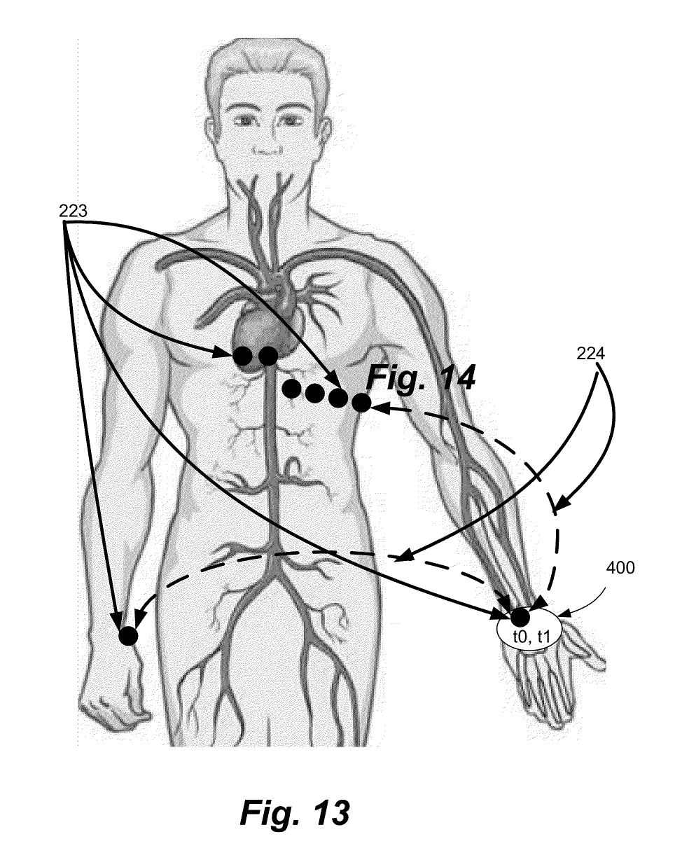

Some of the electrical signals that accompany heart contractions are measurable at the wrists and torso. These signals may be detected and used by electrocardiographs to construct EKG waveforms. Some of the locations 223 at which these signals are measurable, given a reference voltage, are shown in FIG. 13. Electrical potential, or voltage differences 224, between the wrist and torso or between wrists, are differential signals and hence do not require a reference voltage level. Since the transmission of the electrical signals of the heart to various remote detection points on the body is independent of blood viscosity, the timing of these signals may be used to calculate time t0 when the heart generates a particular pulsatile wave.

FIGS. 13-15 depict a wearable blood viscosity measuring device 400 that detects both t0 and t1 at a single location. Similar to the blood viscosity measuring system shown in FIG. 12, device 400 may use two sensors, 408 and 406, to derive respectively, a signal at time t0 when the pulsatile wave is generated, and a signal at time t1 when the pulsatile wave is detected at distance Leq. However, unlike FIG. 12 that depicts a module 350 to detect t0 and a separate module 360 to detect t1, FIGS. 13-15 depict a single device 400 to measure both t0 and t1.

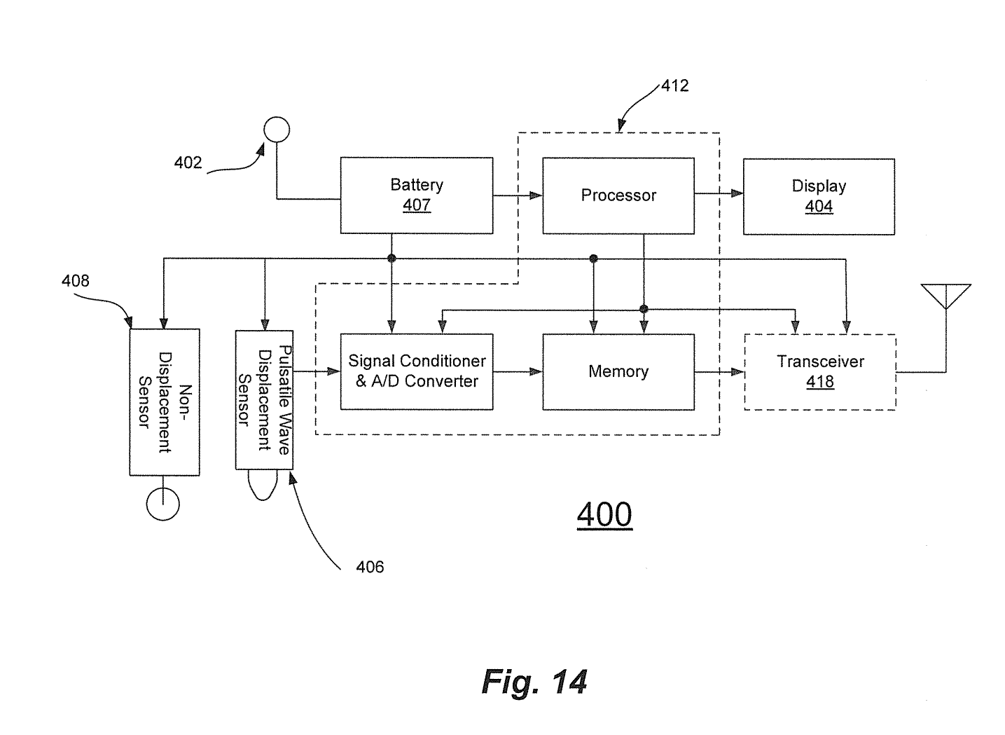

The blood viscosity measuring device 400 shown in FIG. 14 may be worn at a position on the outside of the body where both the bulge formed by the blood viscosity dependent pulsatile wave front, as well as a secondary or related heart signal, not blood viscosity dependent, may be detected. One such location is the wrist area where device 400 may be strapped so as to detect, by sensor 408, at least one EKG type electrical signal associated with a heart contraction to provide time t0, and to detect, using sensor 406, a displacement due to the pulsatile wave to provide time t1. As presented above, knowledge of t0, t1, and distance Leq allows device 400 to calculate a real-time blood viscosity level based upon measurements taken at device 400.

FIG. 14 depicts a block diagram of the device 400 shown in FIGS. 15a-15c. The pulsatile wave generator activity detector 408 may detect electrical potentials or other signals related to the generation of a pulsatile wave by the heart. If an EKG type electrical signal is detected, an electrically conducting contact, for example metallic rim 402, may be used as a reference potential. In normal operation, sensor 408, located on the backside 410 of device 400, may be in contact with the skin. When blood viscosity is to be measured, the user may touch rim 402 with the opposite hand to generate a measurable differential signal related to the time t0 when the pulsatile wave was generated by the heart.

Pulsatile wave sensor 406 may likewise be mounted on the backside 410 of device 400 and may press against the surface of the skin separating sensor 408 from the arterial blood vessel to detect a pulsatile wave and therefore marking time t1.

Pulsatile wave sensor 406 may utilize one of several known technologies to detect the pulsatile wave, including piezoelectric and photoelectric devices. Controller module 412 may use time measurements t0 and t1 provided by sensors 406 and 408 to calculate the blood viscosity level based upon programmed instructions that implement one or more of equations (1)-(9) and may display the calculated blood viscosity value on a display 404, mounted on a front surface 412 of device 400. Display 404 may include an LED display, an LCD display, or any other appropriate display device.

Alternatively, controller module 412 may transmit via transceiver 418, one or more of signals t0, t1, and the calculated blood viscosity value to one or more external devices, such as a smartphone, tablet, or other computing device.

### Additional Embodiments

In addition to measuring blood viscosity, both the wearable and implantable versions of the devices described herein may also measure blood pressure. This is possible because the systolic and diastolic blood pressure values are directly related to features of the full shape of the pulsatile wave bulge 204.

While the embodiments have been described with reference to examples, various modifications to the described embodiments may be made without departing from the scope of the claimed embodiments.

1. A method of calculating a blood viscosity value of blood flowing through a blood vessel comprising:
detecting a deformation of the blood vessel due to a pulsatile wave of blood flowing through the blood vessel;
determining a pulsatile wave velocity of the pulsatile wave based upon the detected deformation; and
calculating the blood viscosity value of the blood flowing through the blood vessel based on a predetermined relationship between blood viscosity and the determined pulsatile wave velocity.

2. The method of claim 1, wherein determining the pulsatile wave velocity comprises:
detecting the deformation of the blood vessel due to a pulsatile wave of blood flowing through the blood vessel at a first position and at a second position along the blood vessel;
determining a time interval between an arrival of the pulsatile wave at the first position and an arrival of the pulsatile wave at the second position based upon detected deformations of the blood vessel at the first and second positions; and
dividing a distance between the first position and the second position by the determined time interval to determine the pulsatile wave velocity.

3. The method of claim 1, wherein detecting the deformation of the blood vessel comprises:
detecting a shape of the deformation of the blood vessel, and
wherein determining the pulsatile wave velocity comprises determining the pulsatile wave velocity based upon the detected shape of the deformation of the blood vessel.

4. The method of claim 3, wherein detecting the shape of the deformation of the blood vessel comprises taking multiple measurements of a displacement of a wall of the blood vessel at a front of the pulsatile wave as the pulsatile wave front travels though the blood vessel.

5. The method of claim 1, wherein determining the pulsatile wave velocity of blood flowing through the blood vessel is based upon at least one implanted sensor positioned in close proximity to the blood vessel.

6. The method of claim 1, wherein determining the pulsatile wave velocity of blood flowing through the blood vessel includes detecting, by an externally mounted sensor held against the skin of a user whose blood viscosity is being measured, the deformation of the blood vessel through the skin of the user.

7. The method of claim 6, wherein determining the pulsatile wave velocity comprises:
tracking a time at which a signal emitted by the heart of the user is detected;
determining a time difference between the tracked time at which the signal is detected and a time at which the deformation of the blood vessel is detected; and
wherein determining the pulsatile wave velocity of the pulsatile wave comprises dividing an equivalent arterial distance measured from the heart of the user to the externally mounted sensor by the determined time difference to determine the pulsatile wave velocity.

8. The method of claim 7, further comprising determining, during an initial calibration stage, the equivalent arterial distance by:
measuring an initial blood viscosity at a location of the sensor based upon an analysis of blood taken at the location of the sensor;
calculating a velocity of the pulsatile wave based upon the measured initial blood viscosity; and
calculating the equivalent arterial distance based upon the calculated velocity of the pulsatile wave and a determined time difference between the detected deformation of the blood vessel and a detected signal emitted by the heart of the user.

9. The method of claim 7, further comprising detecting the signal emitted by the heart of the user by measuring an audible or electrical characteristic of activity of the heart.

10. The method of claim 1, wherein detecting the deformation of the blood vessel further comprises subcutaneously detecting the deformation of the blood vessel by at least one implantable sensor.

11. The method of claim 1, wherein detecting the deformation of the blood vessel includes sensing at least one of physical, electrical, and acoustical changes caused by the deformation of the blood vessel.

12. A blood viscosity measurement and monitoring system, comprising:
at least one sensor to detect a deformation of a blood vessel;
a controller coupled to the at least one sensor to:
receive a signal indicating the detected deformation of the blood vessel from the at least one sensor;
determine a pulsatile wave velocity of blood flowing through the blood vessel based upon the signal; and
calculate a blood viscosity value of the blood flowing through the blood vessel based on a predetermined relationship between blood viscosity and the determined pulsatile wave velocity.

13. The system of claim 12, wherein the at least one sensor includes:
a first sensor to detect a first deformation of the blood vessel due to a pulsatile wave of blood flowing through the blood vessel at a first position along the blood vessel; and
a second sensor to detect a second deformation of the blood vessel at a second position downstream of the first position along the blood vessel;
wherein the controller is to determine a time interval between the first deformation and the second deformation.

14. The system of claim 13, wherein to calculate the pulsatile wave velocity, the controller is to divide a distance between the first position and the second position by the determined time interval.

15. The system of claim 12, wherein the at least one sensor is to be imbedded in a ring implantable within a user, wherein the ring is to surround, in close proximity to, the blood vessel.

16. The system of claim 12, wherein the at least one sensor includes a pulsatile wave displacement sensor to detect the deformation of the blood vessel.

17. The system of claim 12, wherein the at least one sensor includes a sensor to detect an activity related to a generation of the pulsatile wave.

18. The system of claim 12, wherein the at least one sensor detects an audible or electrical characteristic of activity of the heart.

19. A non-transitory computer readable medium comprising machine readable instructions for calculating a blood viscosity level, the machine readable instructions, when executed by a processor, cause the processor to:
detect a deformation of a blood vessel due to a pulsatile wave of blood flowing through the blood vessel;
determine a pulsatile wave velocity of the pulsatile wave based upon the detected deformation; and
calculate the blood viscosity value of the blood flowing through the blood vessel based on a predetermined relationship between blood viscosity and the determined pulsatile wave velocity.

20. The non-transitory computer readable medium of claim 19, wherein to determine the pulsatile wave velocity, the machine readable instructions are further to:
detect a deformation of a blood vessel due to a pulsatile wave of blood flowing through the blood vessel at a first position and at a second position along the blood vessel;
determine a time interval between an arrival of the pulsatile wave at the first position and an arrival of the pulsatile wave at the second position based upon the detected deformations of the blood vessel at the first and second positions; and
dividing a distance between the first position and the second position by the determined time interval to determine the pulsatile wave velocity.
